(19) United States
(12) Patent Application Publication
(10) Pub. No.: US 2009/0222966 A1
(43) Pub. Date: Sep. 10, 2009

(54) Device and Method for Preventing the Obstruction of Eyewear

(76) Inventor: Kelly Tippett, Racine, WI (US)

Correspondence Address: THE LAW OFFICE OF RANDALL T. ERICKSON, P.C., 1749 S. NAPERVILLE ROAD, SUITE 202, WHEATON, IL, 60187, US

(21) Appl. No.: 12/392,767

(22) Filed: Feb. 25, 2009

Related U.S. Application Data
(60) Provisional application No. 61/068,662, filed on Mar. 10, 2008.

(51) Int. Cl. A61F 9/00

(52) U.S. Cl. 2/13

(57) ABSTRACT

A gas curtain device having an air or gas pathway. The air pathway includes a tube with a hole or holes extending a portion of the length of the protective eyewear lens. The pathway is attached or incorporated into an eyewear device. The holes direct air or gas to create an air or gas curtain across the surface of the lens of an eyewear device. The device may have a air or gas pump for directing air into and out of the pathway. The air curtain pushes any debris away from the lens towards the ground and preventing the accumulation of debris on the lens thereby preventing the obstruction of a user's visual field.

This application claims the benefit of U.S. Patent Application No. 61/068,662 filed on Mar. 10, 2008.

### FIELD OF THE INVENTION

The present invention relates to eyewear devices, and particularly relates to devices that prevent obstruction of the lens of an eyewear device.

### BACKGROUND OF THE INVENTION

In certain industries, such as painting, construction, and pest control, workers are subjected to constant fall-out or bounce-back of lightweight particles, especially from spraying techniques. Spraying coats the intended target but also has a tendency to coat the immediate surroundings, including workers and bystanders. As a result, appropriate precautions must be taken to safe guard the health and safety of workers performing the spraying and those in the vicinity. A significant concern is the protection of a worker's eyes, particularly since many materials that are not otherwise hazardous to skin, or for which ordinary clothing provides appropriate protection, are much more hazardous to eyesight, with the potential to cause either temporary or permanent harm. A typical solution to provide eye protection is to use protective eyewear equipment, usually in the form of safety glasses or goggles, which serve as a barrier from toxic and non-toxic materials.

However, goggles and safety glasses have one particular disadvantage: eventually the material being sprayed will coat the lens, which would then need to be cleaned with water, a strong solvent, or simply discarded. Most material being sprayed, such as paint, tends to dry quickly and a strong solvent is usually needed to clean the lens. The solvent, in turn, can create adverse effects on the lens and obscure visibility thus making the safety glasses or goggle useless.

Economically, time and money are wasted when workers pause periodically in the midst of their work in order to clean debris off the lens of their goggle or safety glasses. In addition, the slow accumulation of debris becomes distracting and irritating. As a result, safety glasses or goggles can be inconvenient to use and are often neglected by workers. Whether spraying paint, drywall texture, or chemicals, there is not only a need to protect the eyes, but also the need to do so in a manner that allows for convenient, rapid vision restoration.

A number of attempts have been made to address the problem of clearing debris off the lens of safety glasses or goggles. One attempt, U.S. Pat. No. 7,469,423, and herein incorporated, provides a plurality of superimposed layers of disposable transparent material on the lens of the safety glasses. Each layer can be removed successively as each becomes laden with debris. Practically, there are several drawbacks in using layers of disposable transparent material. One major drawback is that only a few layers of transparent material can be superimposed, typically about five, before the field of vision becomes distorted. Most tasks, such as painting a ceiling, will require at least thirty clean changes of the superimposed layers before the task is completed, which means a user has to replace at least six batches of the transparent material. Another major drawback is the tendency of the debris to accumulate between the translucent layers, making it difficult to remove the layers thus aggravating the problem rather than addressing it.

Another attempt provides the safety glasses or goggle with a transparent film supply and take up system. As the debris accumulates on the lens, a user advances the film to expose a clean surface thus a clear visual field. These devices have generally been proven unsatisfactory due to their cumbersome nature. The supply and take up system tends to be bulky, the motion of advancing the film is difficult when workers wear gloves, as when using toxic chemicals, and the motion of advancing the film tends to move or dislodge the safety glasses requiring a user to readjust the safety glasses each time the film is advanced. Furthermore, as paint or other materials build up within the supply, the device becomes inoperative and the lens soon becomes obscured by debris build-up. Also, replacing the supply can be difficult and cumbersome for the user.

The inventor recognizes it would be desirable to have a device that prevents falling materials, such as paint, dust or any other lightweight liquid or solid materials, from settling on the lens and obstructing a user's field of vision.

### SUMMARY OF THE INVENTION

The present invention provides a method and device that prevents or reduces fall-out or bounce-back of lightweight materials, especially from spray techniques, from accumulating on the lens of protective eyewear devices, such as goggles or safety glasses, and obstructing a user's field of vision.

The device comprises an air or gas pathway that creates an air or gas curtain across at least a portion of the surface of a lens of an eyewear device.

In one embodiment of the invention, the eyewear device is safety glasses that has a first tube or air curtain tube attached to the frame of the safety glasses and extends the length of the frame and lens of the safety glasses. The embodiment also comprises of an air pump, an air filter, a plastic tubing, two reducer fittings, and a baffle.

The air curtain tube can be made of metal or plastic (rubber, polyvinyl chloride (PVC), polyethylene, polyurethanes, etc.). However, a lightweight plastic will make for easier and more comfortable usage. The air curtain tube is configured with holes along the length of the tube. In one embodiment, the pattern or design of the holes may maximize the air curtain that is created when air is expelled from the holes by the air pump. The pattern or design can comprise of one circular hole extending the length of the tube, a series of circular holes staggered along the length of the tube, one slit extending the length of the tube, a series of slits staggered along the length of the tube, or a combination.

The air curtain tube can be made as a permanent attachment to the frame of the safety glasses, i.e. molded to or incorporated into the frame, or releasably attached to the frame by means of hook and loop fasteners, zip ties, or any other type of fastener that can secure the air curtain tube onto the frame and unsecure the air curtain tube from the frame for ease of cleaning or replacement. The air curtain tube is attached, either permanently or releasably, to the frame in a manner that allows the holes to be directed in a downward direction, towards the lens. In one embodiment the hole may direct are downward but angled slightly away from the lens to create an air or gas curtain in front of the lens. The air curtain may come down at an angle to cross over the lens or may come down immediately in front of the lens depending upon the pattern. Also, the air curtain may or may not touch the surface of the lens.

As debris is falling, the air curtain pushes the debris away from the lens and down towards the ground.

The air curtain tube has a closed end and an open end. The open end attaches to a 45° fitting, which also attaches to a second tube. The second tube may be a lightweight plastic tube, which in turn, may be attached to another fitting that functions to increase the diameter of the plastic tube in order to attach it to the air or gas source. A need for a fitting will be dependent upon the size of the inlet of the air or gas source as some plastic tubes may attach to an air or gas source without a fitting.

The air or gas source can be a conventional air pump or gas pump so long as the pump is capable of pumping air or gas through the air or gas pathway to create an air or gas curtain that can effectively push falling debris away from the lens.

The inlet of the air pump is covered by an air filter, which prevents any material or debris that may have entered the air pump from entering the air curtain tube, exiting the air curtain tube, and preventing proper formation of the air curtain. Air pumped from the air pump passes through the plastic tube, into the air curtain tube, and out of the air curtain tube to create a transparent or invisible air barrier over the lens of the safety glasses thus preventing any falling material or debris from settling onto the surface of the lens. The level of transparency or invisibility of the air or gas barrier, as seen by the naked eye, depends upon such variables as temperature and type of gas used.

Numerous other advantages and features of the present invention will be become readily apparent from the following detailed description of the invention and the embodiments thereof, and from the accompanying drawings.

### BRIEF DESCRIPTION OF THE DRAWINGS

FIG. 1 is a perspective view of the disclosed invention including front view of safety glasses, tubings, fittings, and air pump.

FIG. 2 is a front view of the safety glasses and air curtain tube.

FIG. 3 is a first side view of the safety glasses and air curtain tube.

FIG. 4 is a second side view of the safety glasses and air curtain tube.

FIG. 5 is a perspective view of the underside of the air curtain tube and safety glasses.

### DETAILED DESCRIPTION OF THE PREFERRED EMBODIMENTS

While this invention is susceptible of embodiment in many different forms, there are shown in the drawings, and will be described herein in detail, specific embodiments thereof with the understanding that the present disclosure is to be considered as an exemplification of the principles of the invention and is not intended to limit the invention to the specific embodiments illustrated.

The device 20 comprises a gas or air pathway 30, 60 for forming a gas or air curtain across the lens of an eyewear device. The device 20 is used with eyewear devices 10 such as goggles, face shields, safety glasses, sunglasses, and eyeglasses. The eyewear devices may comprise prescription eyewear devices or non-prescription eyewear devices. The device 20 can also easily be used with or incorporated into full or partial face respirators, welding hoods, or other devices that covers a user's eyes. The device 20 may be used with or incorporated into any eye covering device or eye protecting device.

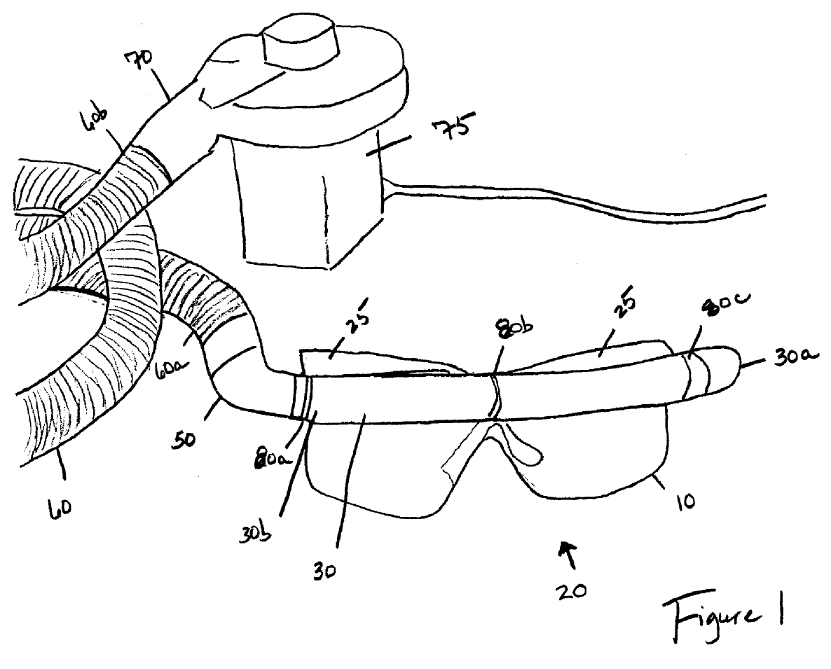

In one embodiment of the invention, the pathway 30, 60 comprises of a first tube 30 and a second tube 60. The first tube 30 can be an air curtain tube, made of metal or plastic (rubber, polyvinyl chloride (PVC), polyethylene, polyurethanes, etc.). However, a lightweight plastic material will make for easier and more comfortable usage. The air curtain tube 30 can be releasably attached to the frame of the eyewear device. In another embodiment, a portion of the air pathway including the air curtain tube 30 is permanently attached by, for example, being molded to or integrated into the frame of the eyewear device. In an embodiment where the air tube is releasably attachable, the air tube is easily replaceable and easily cleaned Also a releasabily attachable air tube may have the veracitility to attach to a variety of protective eyewear devices. The releasabily attached embodiment is shown in FIGS. 1-5.

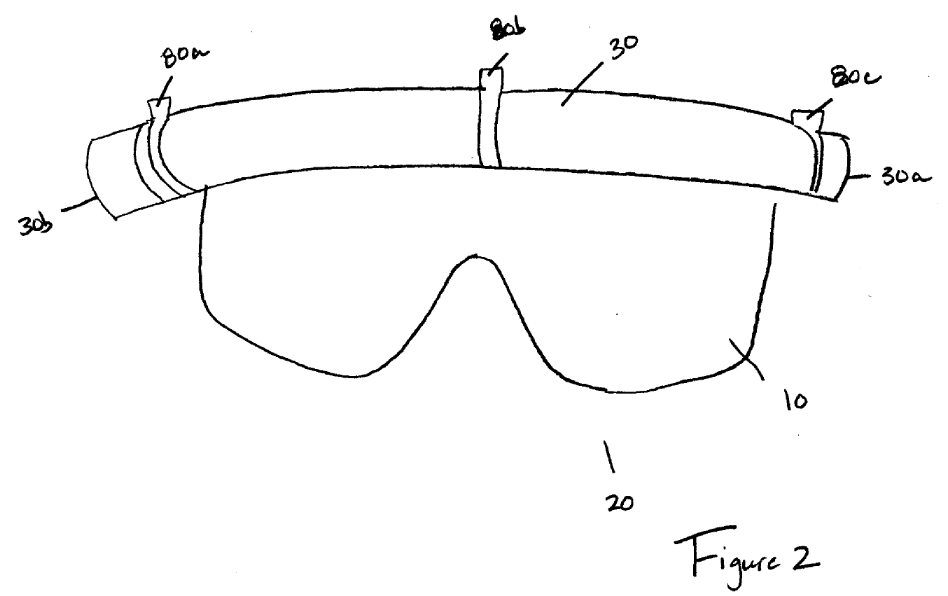

As shown in FIG. 2, the air curtain tube 30 is releasably attached to the upper portion of the frame 25 by means of zip ties 80a, 80ab, 80c at three locations; one in the center 80b, one on the far left 80a, and one on the far right 80c. Other attachment devices, such as hook and loop fasteners, may be used so long as the attachment device can secure the air curtain tube onto the frame 25 and can unsecure the air curtain tube from the frame for easy cleaning or replacement.

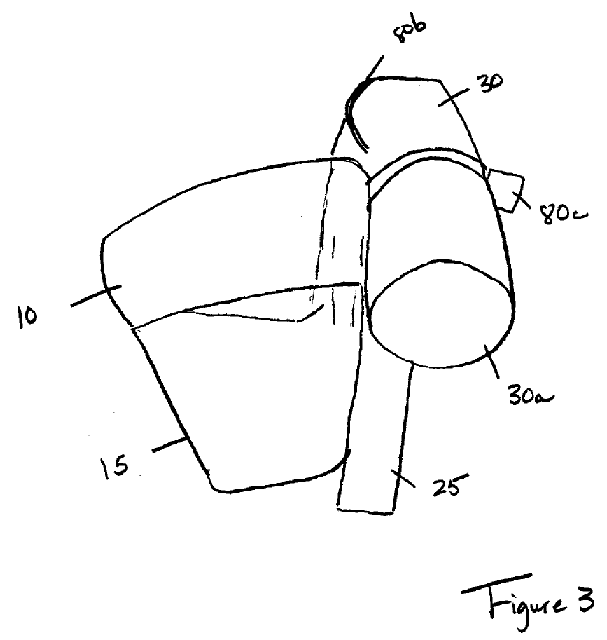

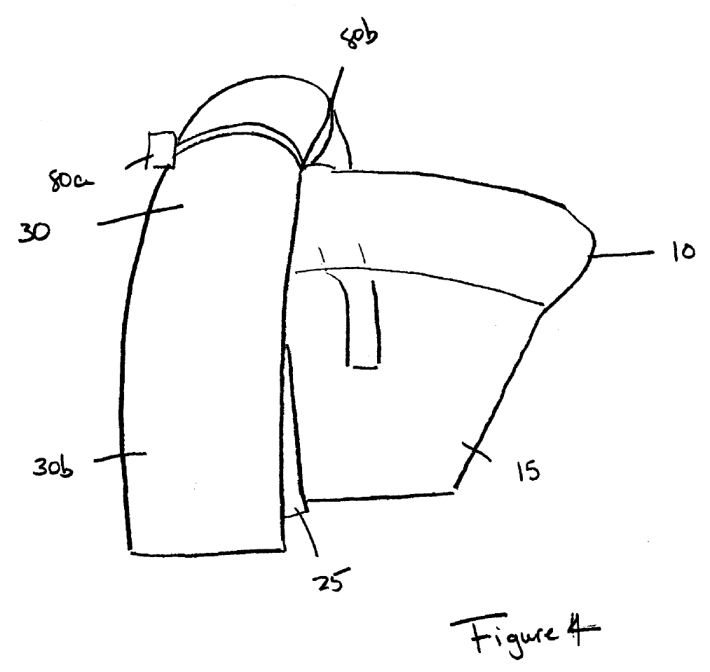

In the illustrated embodiment, the air curtain tube 30 is slightly curved in order to extend the entire length of the frame and lens of the safety glasses, as shown in FIGS. 3 and 4. The air curtain tube 30 has an open-end 30b and a closed-end 30a. The open-end may be located on the right side or left side to accommodate a left-handed or right-handed user, respectively, so that components of the device, such as the second tube 60, do not interfere with a user's movement.

According to FIG. 1, the closed-end 30a is capped or sealed and the open-end 30b has a 45 degree fitting 50 that also attaches to the proximal end 60a of a second tube 60. The second tube can be a lightweight plastic tube 60. The 45 degree fitting 50 can be made of the same material or different material from the air curtain tube 30; making it the same lightweight plastic keeps the invention light and thereby easy and comfortable to use.

The plastic tube 60 may vary in length, depending upon whether the user wishes to carry the plastic tube 60 and the air source 75 by means of a hook or snap attached to a user's pant belt, pant belt loop, or tool belt, or whether a user wishes to place the air source at a specific location within a user's vicinity, which would require a longer plastic tube 60. It is also contemplated that the plastic tube 60 can be an adjustable tube where the length of the tube is variable to accommodate each user's needs.

In one embodiment, the distal end 60b of the second tube 60 is attached to a second fitting 70 that increases the diameter of the plastic tube 60 in order to attach the plastic tube 60 to the air source 75. The need for a second fitting will depend upon the size of the plastic tube 60 and the size of the inlet of the air source 75, as some plastic tubes 60 will easily attach to some air source 75 inlets.

The air or gas source 75 can be a conventional air pump or gas pump so long as the air or gas pump is capable of pumping air or gas through the air or gas pathway to create an air or gas curtain. The source 75 should move air or gas with sufficient force to direct air or gas through the pathway to prevent failing debris from obstructing the lens surface of the eyewear device.

The air pump 75 in FIG. 1 has a air filter (not shown) covering the inlet 73 of the air pump 75 to prevent material or debris from entering the air curtain tube 30 and blowing out of the air curtain tube 30 to either interfere with the formation of the air curtain and/or accumulate on the lens thereby obstruct a user's field of vision.

In another embodiment, there is a baffle plate (not shown), which may be positioned anywhere along the air pathway. The baffle may be placed in the tube 30 to distribute the air or gas flow through holes 40 of the tube 30.

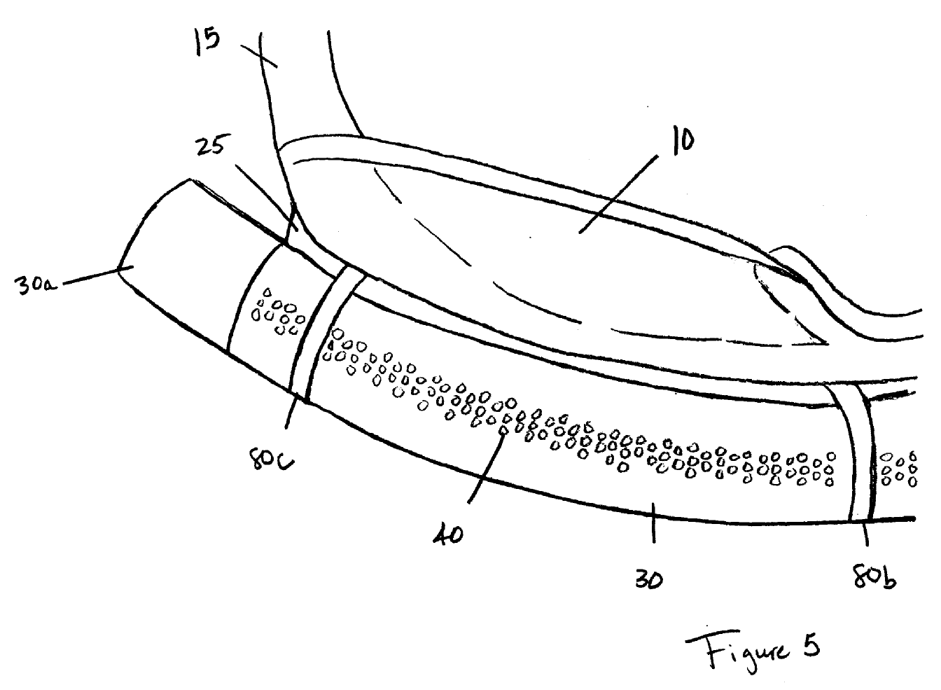

FIG. 5 depicts the underside of the air curtain tube 30. The tube has one or more holes 40, The one or more holes have been formed into the air curtain tube 30 to permit air pumped from the air pump 75 to be released thus creating a curtain of air in front of the lens that prevents sprayed material or debris from accumulating on the lens to obstruct the user's field of vision. When there are more than one hole, the holes may be the same size or of varying size. In one embodiment, the holes 40 form a pattern or design that is meant to maximize the air curtain that is created when air is expelled from the holes 40 by the air pump 75. The pattern or design can comprise of one hole extending along a length of the tube, a series of holes staggered or aligned along the length of the tube, a slit extending the length of the tube, a series of slits staggered or aligned along the length of the tube, or a combination thereof The tube 30 may be positioned, or the holes formed to direct air or gas onto surface of the lens, or to angle air slightly away from the lens to create an air or gas curtain in front of the lens. The air curtain may come down at an angle to cross over the lens or may come down immediately in front of the lens depending upon the pattern. Also, the air curtain may or may not touch the surface of the lens. The power source of the embodiment in FIG. 1 is an external alternating current (AC) power supply; however, the power source can also include batteries, solar cells, or other power sources.

The air pump 75 pumps air through the second tube 60, into the air curtain tube 30, and out of the air curtain tube 30 to create a transparent or invisible air barrier over the lens of the safety glasses thus preventing any falling material or debris from settling onto the surface of the lens. The level of transparency or invisibility of the air or gas barrier, as seen by the naked eye, depends upon such variables as temperature and type of gas used.

From the foregoing, it will be observed that numerous variations and modifications may be effected without departing from the spirit and scope of the invention. It is to be understood that no limitation with respect to the specific apparatus illustrated herein is intended or should be inferred. It is, of course, intended to cover by the appended claims all such modifications as fall within the scope of the claims.

The invention claimed is:

1. A method of preventing material, particles, or debris from obstructing the lens of an eyewear device, comprising the steps of:
forming a gas curtain across the outside surface of the lens.

2. The method of claim 1, wherein the system of forming comprises the step of directing gas over the outside surface of the lens.

3. The method of claim 1, further comprising the step of pumping gas through a pathway that directs gas in front of the outside surface of the lens.

3. The method of claim 1, wherein the forming step comprises the step of forming a gas curtain across the entire surface of the lens.

4. The method of claim 1, comprising the step of preventing the accumulation of material or debris on the lens by the gas curtain.

5. An apparatus for preventing material, particles, or debris from obstructing the lens of an eyewear device, comprising:
a pathway configured to direct gas over an outside surface of the lens.

6. The apparatus of claim 5, comprising a gas source connected to the pathway.

7. The apparatus of claim 5, comprising a pump connected to the pathway for pumping gas into the pathway.

8. The apparatus of claim 5, wherein the pathway comprises a tube, the tube having at least one hole directing gas over the outside surface of the lens.

9. The apparatus of claim 5, wherein at least a portion of the pathway comprises at least a portion of a frame of the eyewear device.

10. The apparatus of claim 5, wherein at least a portion of the pathway is releasably connected to a frame of the eyewear device.

11. The apparatus of claim 5, wherein at least a portion of the pathway is permanently connected to a frame of the eyewear device.

12. The apparatus of claim 7, wherein the pathway comprises a first tube and a second tube; the second tube flexibly connecting the pump to the first tube; the second tube releasably attached to the pump; the first tube having at least one hole directing gas over the outside surface of the lens.

13. The apparatus of claim 7, wherein the pump has a filter.

14. The apparatus of claim 8, wherein the tube is located above the lens and the at least one hole is directed downward and slightly away from the outside surface of the lens.

15. The apparatus of claim 8, wherein the at least one hole comprises a plurality of holes.

16. The apparatus of claim 8, wherein the at least one hole comprises a plurality of holes directing gas across the entire surface of the lens.

17. The apparatus of claim 8, wherein the at least one hole forms a pattern that optimizes the gas forming over the outside of the lens.
